(19) United States
(12) Patent Application Publication
(10) Pub. No.: US 2025/0066741 A1
(43) Pub. Date: Feb. 27, 2025

(54) COMPOSITIONS AND METHODS FOR TRANSFER USING CER1

(71) Applicant: The Trustees of Princeton University, Princeton, NJ (US)

(72) Inventors: Coleen T. MURPHY, Princeton, NJ (US); Rachel KALETSKY, Kendall Park, NJ (US); Rebecca S. MOORE, Philadelphia, PA (US)

(73) Assignee: The Trustees of Princeton University, Princeton, NJ (US)

(21) Appl. No.: 18/292,024

(22) PCT Filed: Jul. 26, 2022
(86) PCT No.: PCT/US2022/038324

§ 371 Date: Jan. 25, 2024

Related U.S. Application Data
(60) Provisional application No. 63/227,572, filed on Jul. 30, 2021.

(51) Int. Cl. C12N 7/00; C12N 15/113

(52) U.S. Cl. CPC C12N 7/00; C12N 15/113; C12N 2310/14; C12N 2800/90

(57) ABSTRACT

Compositions of matter and methods for using the composition of matter may be provided. The method may include providing a virus-like particle (VLP) comprising C. elegans retrotransposon 1 (Cer1) and a heterologous RNA-based agent to an organism and allowing the organism to transfer the heterologous RNA-based agent to a tissue within the organism.

### CROSS-REFERENCE TO RELATED APPLICATIONS

This application claims priority to U.S. Provisional Patent Application No. 63/227,572, filed Jul. 30, 2021, the contents of which are incorporated herein in its entirety.

### TECHNICAL FIELD

This application is related to the transference of information to a target, and specifically to the transference of RNA-based agents using C. elegans retransposon 1 (Cer1) to facilitate the transfer.

### BACKGROUND

The transmission of information across generations through non-genetic means, or transgenerational epigenetic inheritance (TEI), was long thought to be impossible due to the Weismann barrier between the germline and somatic cells, which preserves immortal germ cells in their pristine state. However, recent data from worms suggest that inheritance of stress responses may help animals survive in harsh environments.

To date, no effective method or technique has been devised that would allow the transfer of information, such as RNA-based agents for treatment of a particular condition, to a desired target.

### BRIEF SUMMARY

To overcome the hurdles disclosed above, a method for delivering a RNA-based treatment may be provided. The method may include providing a virus-like particle (VLP) containing C. elegans retrotransposon 1 (Cer1) and a heterologous RNA-based agent to an organism. The method may also include allowing the organism to transfer the heterologous RNA-based agent to a tissue within the organism.

In some embodiments, the VLP may be secreted by an expressing organism. In some embodiments, the VLP may be present in a lysate from a lysed organism. In some embodiments, the VLP is present in a medium.

In some embodiments, the tissue the organism is transferring the RNA-based agent to may include a neuron. In some embodiments, the tissue the organism is transferring the RNA-based agent to may be free of a neuron.

In some embodiments, the heterologous RNA-based agent may include a RNA interference molecule. In some embodiments, the heterologous RNA-based agent may include messenger RNA (mRNA). In some embodiments, the heterologous RNA-based agent may include antisense RNA (asRNA). In some embodiments, the heterologous RNA-based agent may include a RNA aptamer. In some embodiments, the heterologous RNA-based agent may include a peptide (e.g., an RNA-peptide conjugate or complex). In some embodiments, the heterologous RNA-based agent may include a molecule that is not replicated or expressed by the VLP or the organism (which may be, e.g., an animal or plant).

In some embodiments, the RNA-based treatment is a preventative treatment for parasitic nematodes, pathogenic nematodes, or a combination thereof.

In some embodiments, the organism may be allowed to transfer both the heterologous RNA-based agent and a peptide to the tissue.

A VLP may be provided. In some embodiments, the VLP may include C. elegans retrotransposon 1 (Cer1) and a RNA-based therapeutic agent.

An RNA-based therapeutic system may be provided. In some embodiments, the system may include a VLP as disclosed herein, and a pharmaceutically acceptable carrier.

A plasmid system may be provided. The plasmid system may include a first plasmid comprising at least one nucleotide sequence encoding Cer1 and a fluorescent protein. In some embodiments, the first plasmid may also include at least one nucleotide sequence encoding an RNA-based agent. In some embodiments, the system may include a second plasmid that includes at least one nucleotide sequence encoding an RNA-based agent.

### BRIEF DESCRIPTION OF DRAWINGS

FIG. 1 is a schematic of a virus like particle containing Cer1 and a heterologous RNA-based agent.

FIGS. 2A-2C are schematics of C. elegans Cer1 (2A), C. elegans Cer4 (2B) and S. cerevisiae Ty3-1 (2C). In the schematic, LTR=Long Terminal Repeat, PBS=Primer Binding Site, MA=Matrix, CA=Capsid, NC=Nucleocapsid, PR=Protease, RT=Reverse Transcriptase, RH=RNaseH, INT=Integrase, SU=Surface, TM=Transmembrane, and PPT=PolyPurine Tract.

FIG. 3A is a graph of P. aeruginosa PA14 avoidance behavior in naïve animals trained with worm lysate from F2s.

FIG. 3B is a graph of avoidance behavior in naïve worms trained with lysate from F2s grand-progeny of control or P11—trained grandmothers. After lysate exposure, worms were split into groups and tested in 3 different choice assays: E. coli OP50 v. PA14, OP50 vs P. fluorescens Pfl5, or OP50 vs S. marcescens.

FIG. 4 is a graph of F2 worm lysates were fractionated using density-based centrifugation. Fractions 1, 3, and 6 from the gradient were used to train naïve worms, followed by PA14 choice assays.

FIGS. 5A-5B are graphs of choice index for F2 lysate (5A) or virus-like particles (5B) from Cer1 mutant worms, showing such lysate does not induce horizontal memory transfer compared to wild type F2 lysate. Each F2 worm lysate (wild type or Cer1 mutant) were the grand-progeny from control or P11-trained grandmothers. Lysate from wild-type or Cer1 mutant F2 was used to train naïve wild-type animals.

FIGS. 5C-5D are graphs of choice index, where wild-type F2 worm lysate was obtained from the grand-progeny of control or P11-trained grandmothers and used to train naïve recipient Cer1 mutants (5C) or germline-less glp-1 worms (5D) compared to wild-type recipient controls.

FIG. 6 is a schematic of F1 RNAi treatment following control or P11 exposure in PO mothers. Reducing F1 expression of a gene required for initiation of transgenerational inheritance should have no effect on behavior (solid line), while reduced F1 expression of a TEI maintenance gene should eliminate memory in the F1 and subsequent generation (dotted line). F1 knockdown of a gene required for the execution of behavior should affect F1 behavior, but not that of subsequent generations (long dashed line).

FIG. 7 is an illustration of a model of germline-to-soma communication of PA14 avoidance through Cer1.

FIG. 8A is a graph showing C. elegans wild-isolate JU1580 mothers exposed to PA14 lawns (left) or small RNAs (right) learn to avoid PA14 in a choice assay.

FIG. 8B is a graph showing JU1580 mothers exposed to E. coli expressing P11 learn to avoid PA14 after training compared to controls.

FIG. 9 is a graph of PA14 avoidance behavior in wild isolate mothers trained on control bacteria or P11-expressing E. coli.

FIG. 10 is a graph of whole-life RNAi knockdown of Cer1 in N2 and KR314 eliminates P11-induced PA14 learned avoidance.

FIG. 11 is an illustration of a model of horizonal memory transfer via Cer1; horizontal transfer of PA14 avoidance memory occurs when naïve worms are exposed to Cer1's virus-like particles from an animal that has already inherited the memory; uptake of Cer1 induces memory directly in that animal and in four generations of its progeny.

### DETAILED DESCRIPTION

In some embodiments, a method for delivering a RNA-based treatment may be provided. The method may include providing a virus-like particle (VLP) containing C. elegans retrotransposon 1 (Cer1) and a heterologous RNA-based agent to an organism.

As used herein, “heterologous”, “foreign”, and “exogenous” RNA-based agents are used interchangeably and refer to agents that comprise or otherwise utilize RNA that does not occur naturally as part of the plant or animal genome in which it is present, or which is found in a location or locations in the genome that differ from that in which it occurs in nature. Here, the RNA-based agent should not occur naturally as part of the virus-like particle, or as part of the plant or animal genome that is the source of the virus-like particles.

In some embodiments, the organism may be a plant. In some embodiments, the plant may be an agricultural plant. In some embodiments, the agricultural plant may be a cereal grain, such as wheat, barley, corn, sorghum, or oat. In some embodiments, the agricultural plant may be a legume, such as chickpea, green pea, lentil, or soybean.

In some embodiments, the organism may be an animal. In some embodiments, the animal may be a cestode, a nematode, or a trematode. In some embodiments, the animal may be a insect, such as a mosquito, louse, or a species of Cimex. In some embodiments, the animal may be a fish. In some embodiments, the animal may be a mammal, such as a mouse, cat, dog, horse, ape, or human.

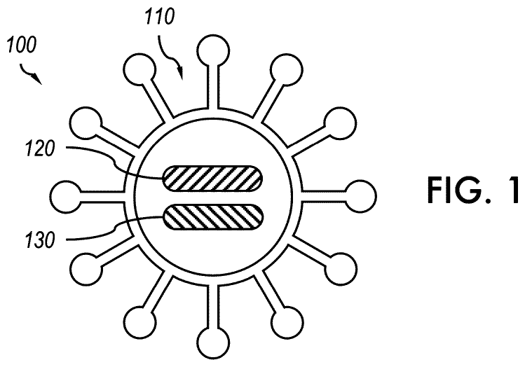

Referring to FIG. 1, the disclosed composition of matter 100 can be seen. The composition 100 includes a virus-like particle 110 containing Cer1 120 and a heterologous RNA-based agent 130.

Virus-like particles (VLPs) are multiprotein structures that mimic the organization and conformation of authentic native viruses but lack the viral genome. A virus like particle is typically composed of one or more viral structural proteins that spontaneously assemble into a particulate structure.

In some embodiments, the VLP may be secreted by an expressing organism. In some embodiments, the VLP may be present in a lysate from a lysed organism. In some embodiments, the VLP is present in a medium, such as a liquid pharmaceutically acceptable carrier.

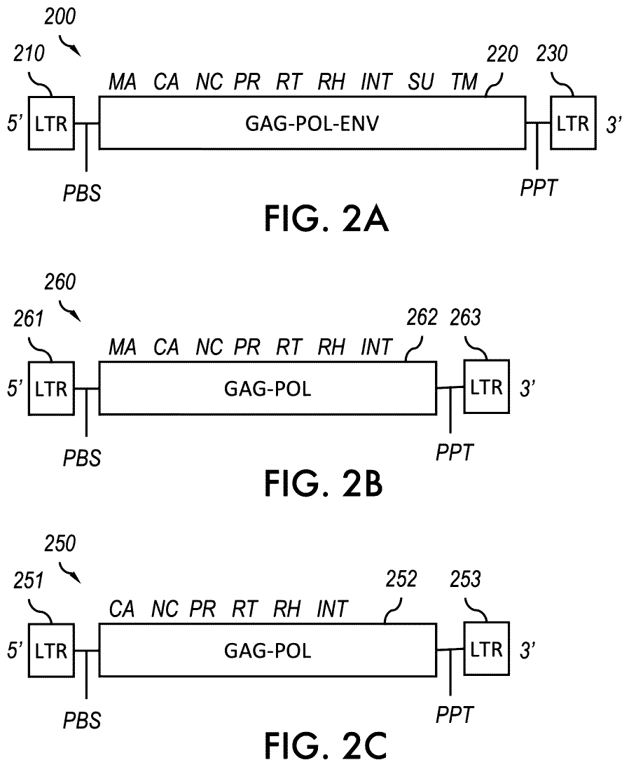

As is known in the art, Cer1 is an 8.8 kb LTR retrotransposon in the Gypsy/Ty3 family of retroviruses/retrotransposons. Referring to FIG. 2A, Cer1 200 has two long terminal repeat (“LTR”) sections 210, 230 on either side of a single, exceptionally long (6819 nt) open reading frame 220 with the potential to encode, e.g., a GAG- and/or POL-containing protein.

Referring to FIGS. 2B and 2C, Cer4 250 and Ty3-1 each have two long terminal repeat (“LTR”) sections 251, 253, 261, 263 on either side of an open reading frame 252, 262 with the potential to encode, e.g., a GAG- and/or POL-containing protein.

As seen in FIGS. 2A-2C, and as understood in the art, “PBS” refers to a primer binding site, “PPT” refers to polypurine tract, “MA” refers to matrix, “CA” refers to capsid, “NC” refers to nucleocapsid, “PR” refers to protease, “RT” refers to reverse transcriptase, “RH” refers to RNAse H, “INT” refers to integrase, “SU” refers to surface, and “TM” refers to transmembrane.

The heterologous RNA-based agent will preferably be an agent configured to target a component in the tissue (e.g., bind to other RNA, interact with a protein, etc.).

In some embodiments, the heterologous RNA-based agent may include a RNA interference (RNAi) molecule. RNAi is a near-ubiquitous pathway that is generally involved in post-transcriptional gene modulation. The key effector molecules of RNAi is generally the microRNA (miRNA) and small interfering RNA (siRNA), which are small, non-coding RNAs transcribed as primary miRNAs and may be processed in the nucleus of a tissue. As used herein, the terms “RNA interference” and “RNAi” are interchangeable and generally refer to the process by which a polynucleotide comprising at least one ribonucleotide unit exerts an effect on a biological process. The process includes, but is not limited to, gene silencing by degrading mRNA, attenuating translation, interactions with tRNA, rRNA, hnRNA, cDNA and genomic DNA, as well as methylation of DNA with ancillary proteins. It is envisioned that any RNAi molecule may be utilized. RNAi molecules are well-known in the art. In some embodiments, the RNAi molecules may be designed to target a nucleic acid sequence that encodes specific proteins that lead to a disease; such sequences are well-documented.

In some embodiments, the heterologous RNA-based agent may include messenger RNA (mRNA). As used herein, the terms “messenger RNA” and “mRNA” are interchangeable and generally refer to a polynucleotide encoding at least one polypeptide. The mRNA may include modified or unmodified RNA as used herein. The mRNA may contain one or more coding and non-coding regions. It is envisioned that any mRNA may be utilized. mRNA is well-known in the art.

In some embodiments, the heterologous RNA-based agent may include antisense RNA (asRNA). As used herein, the terms “antisense RNA” and “asRNA” are interchangeable and generally refer to a single stranded RNA that is complementary to a protein coding messenger RNA (mRNA) with which it hybridizes, thereby blocking its translation into protein. It is envisioned that any asRNA may be utilized. asRNA are well known in the art. For example, mipomersen was approved by the FDA in 2013 for managing the level of low-density lipoprotein cholesterol in certain patients by complementing the mRNA of the protein apo-B-100.

In some embodiments, the heterologous RNA-based agent may include an RNA aptamer. As used herein, “RNA aptamer” refers to RNA oligonucleotides that bind to a specific target with high affinity and specificity, similarly to how an antibody binds to an antigen. Typically, an RNA aptamer may be around 55-120 nucleotides long and may be comprised of a variable region (typically in the center and may be 20-80 nucleotides in length) and at least one constant region (typically on both sides of the variable region, the 5′ and 3′ ends), and may be around 15-20 nucleotides in length. It is envisioned that any RNA aptamer may be utilized. RNA aptamers are well known in the art. For example, numerous RNA aptamers have been isolated, e.g., utilizing the systematic evolution of ligands by exponential enrichment (SELEX) process or a variation thereof.

In some embodiments, the heterologous RNA-based agent may include a peptide connected, conjugated, or otherwise coupled to a heterologous strand of RNA (e.g., an RNA-peptide conjugate or complex).

In some embodiments, the heterologous RNA-based agent may include a molecule that is not replicated or expressed by the VLP (or any material contained in the VLP) or the organism the VLP is intended to be introduced to (which may be, e.g., an animal or plant).

After the VLP containing Cer1 and the heterologous RNA-based agent is provided, the method may also include allowing the organism to transfer the heterologous RNA-based agent to a tissue within the organism. In some embodiments, this may include avoiding the introduction of additional agents, or generating stress in the organism, that may interfere with the transfer.

In some embodiments, the organism is allowed to transfer both the heterologous RNA-based agent and a peptide to the tissue. In some embodiments, the peptide may be non-therapeutic. In some embodiments, the non-therapeutic peptide may be a tagging agent. In some embodiments, the tagging agent may be a fluorophore.

In some embodiments, the RNA-based treatment may be a preventative treatment for parasitic nematode infections. That is, in some embodiments, the RNA-based agent may be an agent configured to treat parasitic nematodes. In some embodiments, the RNA-based treatment may be a preventative treatment for pathogenic nematode infections. In some embodiments, the RNA-based treatment may be a preventative treatment for parasitic nematode infections and pathogenic nematode infections.

In some embodiments, a virus-like particle (VLP) may be provided. The VLP may be configured for delivering a therapeutic agent to a target tissue. Similar to FIG. 1, the VLP may contain C. elegans retrotransposon 1 (Cer1) and an RNA-based therapeutic agent.

The therapeutic agent may be any appropriate therapeutic agent known in the art where the agent functions through transference to a tissue.

The therapeutic agent may mRNA.

The therapeutic agent may be an RNA aptamer, such as pegaptanib, for age-related macular degeneration, which binds specifically to the 165 isoform of VEGF and blocks its function.

The therapeutic agent may be an RNAi. In some embodiments, the RNAi may be a miRNA. In some embodiments, the RNAi may be a siRNA. The siRNA may be patisiran (suppresses hepatic production of transthyretin protein) or givosiran (suppresses hepatic production of ALAS1 protein).

The therapeutic agent may include a peptide, as disclosed herein.

The therapeutic agent may include asRNA, such as fomiversen (binds to IE2 mRNA and blocks translation), mipomersen (binds to ApoB mRNA and induces degradation by RNase H), nusinersen (modulates splicing of SMN2 mRNA and affects the SMN protein level), eteplirsen (induces exclusion of exon 51 of dystrophin mRNA to produce a functional protein), inotersen (binds to transthyretin mRNA and induces degradation via RNase H), or golodirsen (induces exclusion of exon 53 of dystrophin mRNA to produce a functional protein).

In some embodiments, an RNA-based therapeutic system may be provided. The RNA-based therapeutic system may include a VLP as disclosed herein, as well as a pharmaceutically acceptable carrier.

As used herein, “pharmaceutically-acceptable” refers to those compounds, materials, compositions, or dosage forms that are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the VLP, use thereof in the compositions is contemplated. Supplementary active compounds can also be incorporated into the compositions.

In some embodiments, a plasmid system may be provided. The plasmid system will generally include one of two approaches—either a single-plasmid approach, or a multi-plasmid approach.

The first approach is to have a first plasmid that includes at least one nucleotide sequence (such as a heterologous nucleotide sequence) encoding Cer1 and a fluorescent protein (such as green fluorescent protein (GFP)), and also at least one nucleotide sequence (such as a heterologous nucleotide sequence) encoding an RNA-based agent as disclosed herein.

The second approach is to have a first plasmid that includes at least one nucleotide sequence (such as a heterologous nucleotide sequence) encoding Cer1 and a fluorescent protein, and a second plasmid that includes at least one nucleotide sequence (such as a heterologous nucleotide sequence) encoding an RNA-based agent as disclosed herein.

As will be recognized in the art, the plasmids may then be used to transfect one or more organisms, to allow, e.g., the claimed VLPs with Cer1 and heterologous RNA-based agent as disclosed herein to be expressed in the organism.

In some embodiments, a method for delivering a RNA-based treatment may be provided. Any appropriate RNA-based treatment is envisioned here. For example, in some embodiments, the RNA-based treatment is a preventative treatment for parasitic nematodes, pathogenic nematodes, or a combination thereof.

The method may include providing a virus-like particle (VLP) containing C. elegans retrotransposon 1 (Cer1) and a heterologous RNA-based agent as disclosed herein to an organism, and then allowing the organism to transfer the heterologous RNA-based agent to a tissue within the organism. In some embodiments, the organism is allowed to transfer both the heterologous RNA-based agent and a peptide to the tissue.

In some embodiments, the VLP may be secreted by an expressing organism. Thus, in some embodiments, providing the VLP may include growing the expressing organism, allowing it to express the VLP, and collecting the VLP.

In some embodiments, the VLP may be present in a lysate from a lysed organism. Thus, in some embodiments, providing a VLP may include growing the organism, lysing the organism, and collecting the VLP from the lysate.

In some embodiments, the VLP may be present in a medium. For example, in some embodiments, the VLP may be suspended or dispersed in a medium ahead of being used in a treatment.

In some embodiments, the tissue into which the RNA-based agent is transferred may include a neuron. In some embodiments, the tissue into which the RNA-based agent is transferred may be free of a neuron.

### Example

As is known in the art, C. elegans is initially attracted to Pseudomonas aeruginosa, but learns to avoid this pathogen after exposure. Worms learn to avoid P. aeruginosa (PA14) through several independent mechanisms involving bacterial small RNAs, metabolites, and additional pathogenesis factors. However, small RNA-mediated learned avoidance is the only pathway that leads to transgenerational memory inheritance.

C. elegans passes small RNA-mediated learned Pseudomonas aeruginosa avoidance behavior on to several generations of progeny through a molecular mechanism that requires an intact germline and neuronal signaling. This process requires uptake of a P. aeruginosa small RNA called P11, processing through the RNA interference pathway, piRNA regulation and P granule function in the germline, downregulation of a neuronal gene with complementarity to a specific bacterial small RNA, and gene expression changes in the ASI sensory neuron. This small RNA-mediated process enables mothers and four generations of her progeny to avoid pathogenic Pseudomonas aeruginosa.

To test whether transgenerational learned avoidance can be horizontally transferred to naïve worms, mechanical homogenization was used to prepare crude lysates from wild-type grand-progeny (F2) of P11- or control-trained grandmothers.

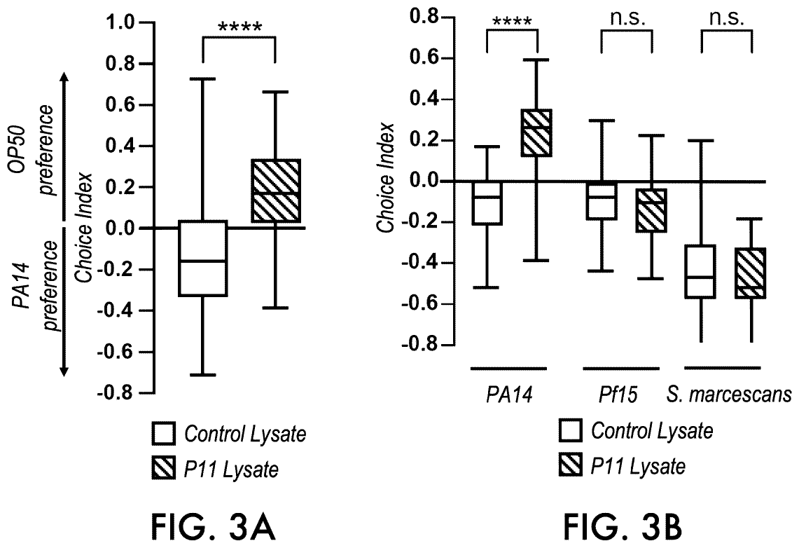

Naïve animals were exposed to the lysate on E. coli plates for 24 h, then tested for P. aeruginosa avoidance learning. We found that lysate from F2s of P11-trained, but not control-trained grandmothers, was sufficient to induce naïve worms to avoid P. aeruginosa (see FIG. 3A), indicating horizontal transmission of memory.

General worm maintenance: Worm strains were maintained at 20° C. on High Growth Media (HG) plates (3 g/L NaCl, 20 g/L Bacto-peptone, 30 g/L Bacto-agar in distilled water, with 4 mL/L cholesterol (5 mg/mL in ethanol), 1 mL/L 1M CaCl2), 1 mL/L 1M MgSO4, and 25 mL/L 1M potassium phosphate buffer (pH 6.0) added to molten agar after autoclaving) on E. coli OP50 using standard methods.

RNAi worm maintenance: For all experiments using control or Cer1 RNAi treated worms had been cultured on HG plates (supplemented with 1 mL/L 1M IPTG, and 1 mL/L 100 mg/mL carbenicillin) for at least three generations, never starving worms.

General bacterial cultivation: OP50 and P. aeruginosa PA14 were cultured overnight in autoclaved and cooled Luria Broth (10 g/L tryptone, 5 g/L yeast extract, 10 g/L NaCl in distilled water) shaking (250 rpm) at 37° C. E. coli strains expressing PA14 small RNAs were cultured overnight shaking (250 rpm) at 37° C. in Luria Broth supplemented with 0.02% arabinose w/v and 100 mg/mL carbenicillin. E. coli RNAi strains were cultured overnight shaking (250 rpm) at 37° C. in Luria Broth supplemented with filter sterilized 12.5 mg/mL tetracycline and 100 mg/mL carbenicillin.

Worm preparation: Eggs from young adult hermaphrodites were obtained by bleaching and subsequently placed onto HG plates seeded with E. coli OP50 or HG RNAi plates seeded with RNAi and incubated at 20° C. for 2 days. Synchronized L4 worms were used in all training experiments.

Bacteria lawn training plate preparation: Overnight cultures of bacteria (prepared as described above) were diluted in LB to an Optical Density (OD₆₀₀)=1 and used to fully cover Nematode Growth Media (NGM) ((3 g/L NaCl, 2.5 g/L Bacto-peptone, 17 g/L Bacto-agar in distilled water, with 1 mL/L cholesterol (5 mg/mL in ethanol), 1 mL/L 1M CaCl2), 1 mL/L 1M MgSO4, and 25 mL/L 1M potassium phosphate buffer (pH 6.0) added to molten agar after autoclaving) plates. For preparation of E. coli expressing PA14 P11 RNA, bacteria were seeded on NGM plates supplemented with 0.02% arabinose and 100 mg/mL carbenicillin. All plates were incubated for 2 days at 25° C. unless specified otherwise (in separate incubators for control/pathogen seeded plates). On the day of training (i.e., 2 days post bleaching), plates were left to cool on a benchtop for 1 hr to equilibrate to room temperature before the addition of worms. Additionally, for E. coli strains expressing PA14 small RNAs, 200 mL of 0.01% arabinose was spotted onto seeded training plates 1 hr prior to use.

Small RNA training plate preparation: 200 μL of OP50 was spotted in the center of a 10 cm NGM. Plates were incubated at 25° C. for 2 days. 100 g of small RNA was placed directly onto OP50 spots and left to completely dry at room temperature (˜1 hr) before use on day of experiment for worm training.

Worm preparation for training: Synchronized L4 worms were washed off plates using M9 and left to pellet on the bench top for approximately 5 minutes. 5 mL of worms were placed onto small RNA-spotted training plates, while 10 mL or 40 mL of worms were plated onto OP50 or E. coli expressing PA14 small RNAs, or pathogen-seeded training plates, respectively. Worms were incubated on training plates at 20° C. in separate containers for 24 hr. After 24 hr, worms were washed off plates using M9 and washed an additional 3 times to remove excess bacteria. Worms were tested in an aversive learning assay described below.

Aversive learning assay: Overnight bacterial cultures were diluted in LB to an Optical Density (OD₆₀₀)=1, and 25 mL of each bacterial suspension was spotted onto one side of a 60 mm NGM plate and incubated for 2 days at 25° C. After 2 days assay plates were left at room temperature for 1 h before use. Immediately before use, 1 mL of 1M sodium azide was spotted onto each respective bacteria spot to be used as a paralyzing agent during choice assay. To start the assay (modified from Zhang 2005), worms were washed off training plates in M9 allowed to pellet by gravity, and washed 2 additional times in M9. 5 mL of worms were spotted at the bottom of the assay plate, using a wide orifice tip, midway between the bacterial lawns. Aversive learning assays were incubated at room temperature for 1 hr before manually counting the number of worms on each lawn. Plating a large number of worms (>200) on choice assay plates was avoided, since excess worms clump at bacterial spots making it difficult to distinguish animals, and high densities of worms can alter behavior.

In experiments in which each generation was treated with RNAi: Animals were washed off plates with M9 at Day 1 of adulthood. A subset of the pooled animals was subjected to an aversive learning assay, while the remaining worms were bleached to obtain eggs, which were then placed onto HG or HG RNAi plates and left at 20° C. for 3 days before the next generation was tested.

Statistical analysis of choice assay data: Populations of worms were raised together under identical conditions and were randomly distributed into treatment conditions. Trained worms were pooled and randomly chosen for choice assays. For all choice assays, each dot represents an individual choice assay plate (about 10-300 worms per plate) with all data shown from at least 3 independent replicates. Plates were excluded that contained less than 10 total worms per plate. The box extends from the 25th to the 75th percentile, with whiskers from the minimum to the maximum values. All figures shown pooled data from independent experiments. Statistics were generated using Prism 8. Counting of worms on choice assay plates was performed blind with respect to worm genotype and training condition.

Preparation of bacteria for RNA isolation: Bacteria for RNA collection were prepared as described for training plates (i.e., 2 days on plates at 25° C.). Bacterial lawns were collected from the surface of NGM plates using a cell scraper. Briefly, 1 mL of M9 buffer was applied to the surface of the bacterial lawn, and the bacterial suspension following scraping was transferred to a 15 mL conical tube. PA14, from 10 plates or OP50 from 15 plates was pooled in each tube and pelleted at 5,000×g for 10 minutes at 4° C. The supernatant was discarded and the pellet was resuspended in 1 mL of Trizol LS for every 100 μL of bacterial pellet recovered. The pellet was resuspended by vortexing and subsequently frozen at −80° C. until RNA isolation.

Bacteria RNA isolation: To isolate RNA from bacterial pellets, Trizol lysates were incubated at 65° C. for 10 min with occasional vortexing. Debris was pelleted at 7000×g for 5 min at 4° C. The supernatant was transferred to new tubes containing ⅕ the volume of chloroform. Samples were mixed thoroughly by inverting and centrifuged at 12000×g for 10 min at 4° C. The aqueous phase was used at input for RNA purification using the mirVana miRNA isolation kit according to the manufacturer's instructions small RNA (<200 nt) isolation. Purified RNA was used immediately or frozen at −80° C. until further use as previously described in Kaletsky 2020.

C. elegans total RNA isolation: F2 worms from trained grandmothers were washed off of plates using M9. Three additional M9 washed were performed to remove excess bacteria, and the supernatant was discarded. 1 mL of Trizol LS was added per 100 μl of worm pellet. Worms were lysed in Trizol by incubation at 65° C. for 10 min with occasional vortexing. RNA was extracted with chloroform, and the aqueous phase was used as input for RNA purification using the mirVana miRNA isolation kit according to the manufacturer's instructions for total RNA. Approximately 100 μg of total RNA from either control or P11 grandmother-trained F2 worms was used per training plate. This amount of RNA was chosen as it correlates to the same input of worms used for training with worm lysate (see Preparation of Worm Lysates). Purified RNA was used immediately by dropping RNA onto pre-seeded spots of OP50 on NGM plates. Plates were allowed to air dry before the addition of naïve worms for training. Worms were trained on RNA-seeded plates for 24 h at 20° C. and subsequently tested for PA14 aversive learning using a standard choice assay.

Preparation of worm lysates: Day 1 F2 progeny from control or P11-trained grandmothers were collected from plates and washed 3 times in M9. The worm pellet was washed with DPBS, and the pellet was resuspended in DPBS. Worms were homogenized using an all-glass Dounce tissue grinder (Kimble #885300-0002), and homogenization was monitored using a microscope. Different worm lysates within an experiment were normalized to the starting amount of worms. For training naïve worms with lysates from F2 animals, the normalized lysate was diluted 1:3 with DPBS, such that 400 μl of lysate was obtained for every 100 μl of starting worm pellet. 150 μl of lysate was immediately pipetted directly onto the bacterial spot of 10 cm NGM plate (seeded with 200 μl of an OP50 spot in the center of the plate, 2 days prior to the experiment). Worm lysates were allowed to air dry, and plates with lysates were monitored to ensure no worms were alive following homogenization. Naïve Day 1 worms were then transferred to lysate-seeded plates for 24 h of training at 20° C., followed by testing for learned avoidance using the standard OP50 v. PA14 choice assay.

Training of mothers with either P. aeruginosa or P11 small RNA induces a memory of learned avoidance that lasts through the F4 generation. While the lysate from F2-F4 progeny can induce learning in naïve animals, lysate from the F5 generation—which does not show inheritance of learned behavior from either P. aeruginosa or P11 training—is not able to transfer learned avoidance. Furthermore, progeny of lysate-trained PO animals inherited this learned avoidance behavior, lasting through the F4 generation after training, indicating that transgenerational inheritance can occur after horizontal transfer of memory.

Training animals on P. aeruginosa or P11 small RNA induces avoidance specifically against P. aeruginosa, rather than to other bacteria. To test whether the horizontally-acquired memory is specific to P. aeruginosa, lysate-trained animals were tested for changes in preference to Pseudomonas fluorescens (Pfl5) or Serratia marcescens. While worms exposed to lysate from grand-progeny of P11-trained grandmothers learned to avoid P. aeruginosa compared to controls, lysate training did not alter the worms' attraction to either P. fluorescens (Pfl5) or S. marcescens (See FIG. 3B). These results indicate that the horizontally-transferred memories are specifically encoded for P. aeruginosa avoidance and are likely not caused by a non-specific response that induces broad neuronal changes in preference.

RNA has been implicated in the transfer of memory from the CNS of trained Aplysia to naïve animals. After testing 1) whether free, total RNA isolated from F2s of trained animals could transfer memory, and 2) whether the trained F2 lysate would still transfer memory if treated with RNase before worm training, it was determined that total RNA from trained F2s was not able to induce avoidance learning, and RNase treatment of the trained F2 lysate did not abolish memory transfer.

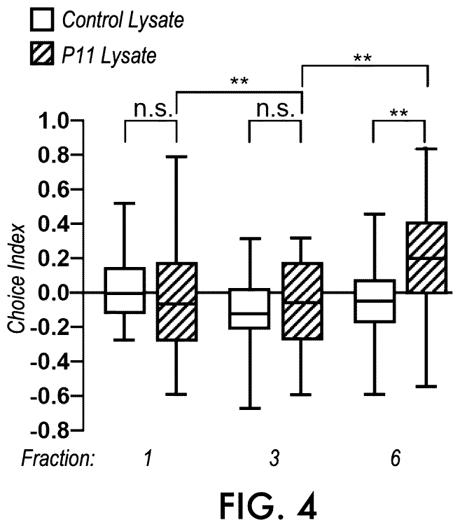

To determine if purified capsids might carry the memory of P11 training, density-fractionated lysates from F2s of P11-trained grandmothers were tested for their ability to induce avoidance. Only the densest fraction (#6), which should contain VLPs, induced P. aeruginosa avoidance behavior in naïve animals. See FIG. 4.

Cer1-enriched fraction isolation: Homogenates were prepared as described (Preparation of worm lysates) and cleared from debris by a 750×g centrifugation at 4° C. for 5 minutes. Homogenization and clearing steps were repeated twice. The homogenates were then passed twice through a 0.22 um filter. For each sample, the homogenate protein concentration was measured using Quant-iT Protein Assay Kit (Invitrogen #Q33211). Per experiment, if needed, the homogenates were diluted in DPBS in order to load similar concentrations. From each sample, a small aliquot was kept as a “load” sample, and 830 uL was layered on top of an Iodixanol gradient. For each gradient-5%, 11%, 17%, 24% and 30%—Iodixanol solutions were made by mixing solution A (0.1 M NaCl, 0.5 mM EDTA, 50 mM Tris HCl, pH 7.4) with solution B [50% Iodixanol solution (OptiPrep, Sigma #D1556), 0.5 mM EDTA, and 50 mM Tris HCl, pH 7.4]. The gradient was made in a 5 mL, Open-Top Thinwall Ultra-Clear Tube (Beckman Coulter #344057) from equal volumes (830 uL) of each Iodixanol solution that were allowed to diffuse by an overnight incubation at 4° C. Samples were then centrifuged at 112,000×g (4° C.) for 2 hours, using SW55 Ti Swinging-Bucket Rotor (Beckman Coulter). Six fractions of equal volumes were collected. Cer1-enriched fraction (fraction 6), as well as fraction 3, were diluted in DPBS and centrifuged at 335,000×g (4° C.) for 30 minutes. Each pellet was then resuspended in DPBS and used for Western blots, naïve worm training, or electron microscopy. For each experiment, the enrichment of Cer1 in fraction 6 was verified by western blot. For fractions treated with RNase, 1:1000 RNaseA (omega BIO-TEK #AC117) was added following resuspension after the final spin, and samples were incubated for 15 minutes at room temperature. For behavior experiments with RNase-treated samples, the reaction was terminated by adding RNase inhibitor (Invitrogen #AM2696, 1 unit final).

To determine whether capsids or virus-like particles were present in the fraction that induced learning behavior, electron microscopy was performed on the densest fraction, and ˜90 nm virus-like particles (VLPs) were identified. This fraction was also able to induce behavior not only in the trained generation, but also through the F4 generation. Although there was too little material to successfully build a library for RNA-seq, the Bioanalyzer trace of the RNase-treated VLPs shows that there is RNA inside the capsids, and RNase treatment of the VLP fraction did not prevent the induction of avoidance learning, supporting the model that capsids protect cargo RNA.

The capsids observed by EM were similar in size to VLPs made by the Cer1 retrotransposon. Cer1 has homology to the Ty3/Gypsy retrotransposon (see FIGS. 2A-2C), and forms VLPs that are detectable by EM and present in the germline of N2 animals at 20° C.

Therefore, it was investigated whether Cer1 might be involved in learned pathogen avoidance and its inheritance. The Cer1 GAG protein was detected in the densest fraction (#6), which induced learned avoidance in wild-type worms. A point mutation (G6369A) in Cer1 abolishes its detection by immunofluorescence or by Western blot, suggesting that this mutation prevents expression of Cer1 gene products. Cer1 #mutant mothers were still able to learn on a P. aeruginosa lawn, consistent with intact routes of lawn learning, such as innate immunity and metabolites; however, loss of Cer1 abolishes the F1 inheritance of P. aeruginosa avoidance behavior, which functions through the separate small RNA-mediated pathway. Reduction of Cer1 via RNAi also abrogated P. aeruginosa-mediated pathogen avoidance inheritance. Loss of Cer1 by mutation or RNAi also completely abrogated the ability of mothers trained on E. coli+P11 to learn P. aeruginosa avoidance. Unlike Cer1, loss of a different Ty3/Gypsy retrotransposon, Cer4, had no effect on learning or transgenerational memory induced by PA14 lawn or E. coli+P11 training of N2 mothers.

Upon training with P. aeruginosa or P11 small RNA, daf-7p::gfp expression increases in the ASI sensory neuron. Loss of Cer1 prevents this increase in expression, suggesting that Cer1 acts upstream of the regulation of daf-7 expression in the ASI neuron. Together, these results suggest that Cer1 is required for small RNA-mediated pathogen avoidance in mothers and their progeny, is present in the VLP fraction that induces learning, and acts upstream of neurons in the small RNA-mediated learning pathway.

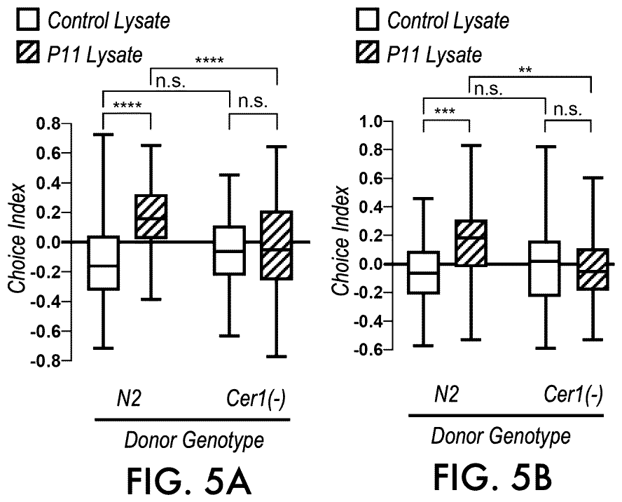

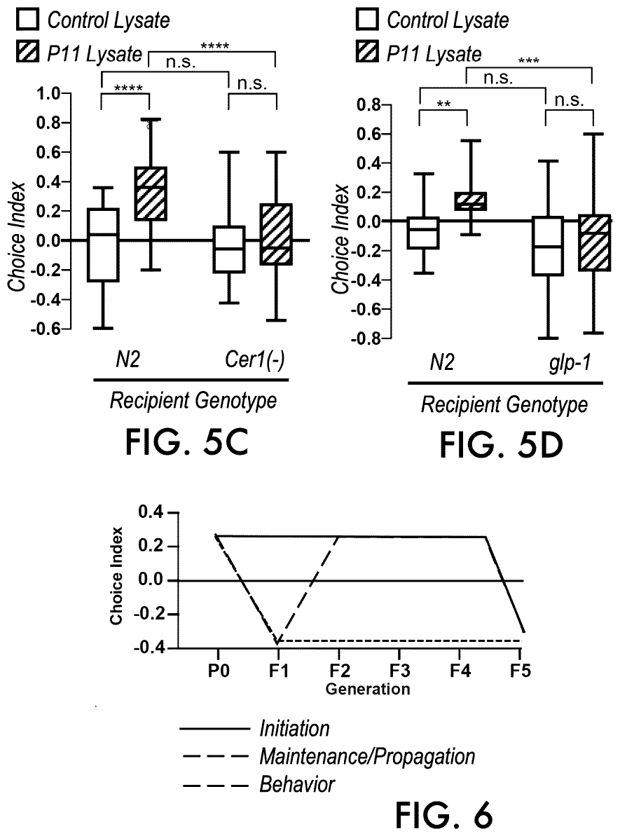

To determine whether Cer1 is required for not only vertical memory transmission to progeny, but also for horizonal memory transfer, worm lysates and VLP-containing fractions from wild type and Cer1 mutant F2s from control or P11-trained grandmothers were prepared as described previously. Consistent with the requirement for Cer1 in horizontal memory acquisition, neither the lysate nor the analogous density-purified fraction (fraction #6) isolated from Cer1 mutants derived from P11-trained grandmothers were able to induce avoidance of P. aeruginosa. See FIGS. 5A-5B. These results suggest that Cer1 capsids are required for the horizontal transfer of transgenerational epigenetic memories to naïve worms.

Since Cer1 is required for both vertical and horizontal transfer of pathogen avoidance learning, it was next asked whether Cer1 and/or a germline is required in recipient worms, or if treating with Cer1-containing lysate bypasses the requirement for Cer1 in recipient animals (for example, by direct uptake by neurons). Cer1 mutants trained with wild-type F2 lysates were unable to learn P. aeruginosa avoidance (see FIG. 5C). Germline-less glp-1(e2141) also failed to learn P. aeruginosa avoidance upon F2 lysate training (see FIG. 5D). These results show that both Cer1 and a functional germline are required in recipient animals for horizontal memory transfer through Cer1 capsid.

These results show that Cer1 is required in mothers for small RNA-mediated learned avoidance and in their progeny for the inheritance of this behavior. It has been found that the process of inducing transgenerational inheritance of pathogen avoidance requires uptake of small, non-coding RNAs from Pseudomonas, processing of this small RNA in the intestine and germline, and transmission of an unknown signal that is conveyed to the ASI neurons to influence avoidance behavior.

To determine the mechanism of Cer1's function in learned pathogen avoidance and its inheritance, it was desirable to determine the step at which it is required—the initiation of the transgenerational signal, maintenance of this signal in the germline from generation to generation, or a subsequent, post-germline step that results in execution of avoidance behavior (transmission of the signal from germline to neurons or neuronal function). The step at which Cer1 acts in the pathway was not clear from initial experiments, because a mutant or Cer1 RNAi for several generations would not distinguish a lasting and permanent effect of Cer1 activation from a transient effect that only affects one step of the transgenerational learned pathogen avoidance process.

However, these steps can be distinguished through a simple experiment: knockdown of the gene of interest in the F1 generation after PO training, followed by control RNAi in generations F2-F5. Knockdown of a gene involved in initiation (PO) would have no effect if reduced only in the F1 generation (see FIG. 6, solid line); F1 knockdown of a gene involved in germline maintenance or propagation would permanently eliminate learned behavior (see FIG. 6, dotted line, “maintenance/propagation”); and F1 knockdown of a gene that only functions in transmission of the signal or functions in neurons would eliminate the behavior for a generation or two, but should return once the RNAi knockdown is ended (see FIG. 6, long dashed line, “behavior”).

Knockdown of sid-2, the NA transporter that is expressed in the intestine, only in F1 does not affect behavior in any generation, likely because its role is to facilitate uptake of bacterial small RNAs from the gut, which is critical in initiation (PO) but is not needed in later generations. By contrast, knockdown of the piRNA Piwi/Argonaute PRG-1 in the F1 generation eliminates behavior not only in F1, but also causes a permanent loss of avoidance behavior. These results are consistent with previous data suggesting that prg-1 is required for maintenance or propagation of avoidance behavior, and that loss of prg-1 erases transgenerational memory.

The TGF-beta ligand DAF-7 is expressed in the ASI neuron, and is required to execute the avoidance behavior. Reduction of daf-7 by RNAi in the F1 generation following maternal P. aeruginosa or E. coli+P11training abrogated avoidance behavior in the same generation (F1). However, progeny raised on control RNAi recovered their avoidance behavior in the F2-F4 generations, demonstrating that the encoded memory was retained even when daf-7expression was reduced, and that avoidance behavior could return. This shows that daf-7 is not required for germline maintenance of transgenerational memory, but is instead involved in the execution of avoidance behavior. These sid-2, prg-1, and daf-7 RNAi initiation vs maintenance vs execution behavior results, respectively, agree with their previously-determined roles in intestine, germline, and neurons.

Cer1 capsids are present in the germline, and their presence depends on prg-1 and P granules in worms; in yeast, Ty3 VLP formation is similarly dependent on P-bodies.

Therefore, it was first hypothesized that Cer1 might function at the step of maintaining the transgenerational signal in the germline, similar to prg-1. However, while Cer1 (RNAi) treatment in the F1 progeny of wild-type mothers trained with E. coli expressing P11 or with P. aeruginosa caused loss of avoidance behavior, the avoidance memory recovered in subsequent generations maintained on control RNAi allowing Cer1 re-expression. These results resembled daf-7 knockdown and recovery, rather than the permanent loss of learned avoidance that prg-1 knockdown causes, suggesting that Cer1 acts in the execution of avoidance behavior rather than at the step of maintenance of the transgenerational signal. This further suggested that Cer1's role in learned pathogen avoidance might not be restricted to germline function, despite the fact that it is primarily expressed in the germline, but rather may act at a step between germline and neuron function.

To test the notion that Cer1 might act in a post-germline, dynamic, transient step, RNAi was carried out starting in adulthood. First, knockdown of Cer1 in trained PO adults blocked avoidance learning as well as whole-life RNAi treatment did, showing that Cer1 can be knocked down effectively in adults. Similarly, loss of Cer1 only in adults prevents the induction of daf-7p::gfp expression in the ASI. Knockdown of Cer1 in trained PO adults followed by treatment on control RNAi in F1 allowed the re-emergence of avoidance behavior, further establishing that Cer1 is not involved in establishment of the transgenerational signal. Knockdown of Cer1 only in adults of the F2 generation abrogated behavior, despite the F1 animals having demonstrated inheritance of avoidance. Together, these results suggest that the process is dynamic: if the transgenerational inheritance of avoidance had been set by regulation of neuronal gene expression levels in the embryonic state, then knockdown of Cer1 should not have affected behavior. Instead, we see that Cer1, which acts upstream of daf-7 in the ASI, dynamically regulates behavior in adult animals.

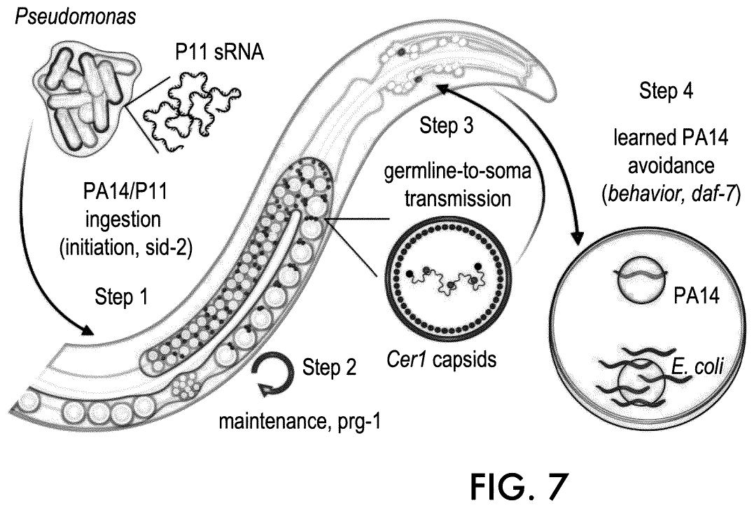

Together, these results show that loss of Cer1 does not erase transgenerational memory, but rather is required downstream of the memory maintenance machinery in order to execute avoidance behavior. Thus, its role is unlikely to be solely in the germline, but more likely in the communication of the status of avoidance state information from the germline to the neurons in every generation. This germline-to-soma signaling (see FIG. 7) ultimately affects neuronal activity and behavior to avoid a common pathogen, and also improves their survival on that pathogen. Together, these functions might provide an evolutionary benefit from the insertion and activity of a retrotransposon that was previously thought to be solely deleterious.

The ability of wild strains of C. elegans to carry out small RNA-induced pathogen avoidance learning and transgenerational memory correlates with Cer1 expression.

Roughly 15% of the C. elegans genome consists of transposon genetic material. The Ty3/Gypsy family retrotransposon Cer1 is one of these elements, and is inserted into the genomes of roughly 70% of wild C. elegans strains, although the sites of these insertions differ —some are present in the pig-1 locus, which regulates “plugging” upon mating, while others are present elsewhere (see Table 1). Similarly, some Cer1 insertions are only remnants of the active transposon, with only LTRs (long terminal repeats) detectable (see Table 1). Therefore, it was wondered whether the presence of full-length Cer1 in the genomes of strains isolated from the wild is necessary or sufficient to confer the ability to learn and remember pathogen avoidance.

TABLE 1
Characterizations of C. elegans wild isolates for plugging, presence and expression
of Cer1, naïve PA14 attraction, and P11 small RNA-induced learning.

| | | | | | | | P11- |
|---|---|---|---|---|---|---|---|
| Wild- | | LTRs in | Cer1 in | Cer1 by | Cer1 by | Normal | induced |
| isolate | Plugging | genome | genome | rtPCR | IF | Attraction | learning |
| N2 | − | + | + | + | + | + | + |
| JU1580 | + | + | + | + | + | + | + |
| DH424 | − | + | + | + | + | + | + |
| KR314 | + | + | + | + | + | + | + |
| ED3040 | + | + | + | + | + | − | − |
| MY2 | + | + | + | + | − | + | − |
| ED3077 | + | + | + | +/− | − | + | − |
| CB4856 | + | + | − | − | − | − | − |
| JU363 | + | + | − | − | − | − | − |
| JU322 | + | − | − | − | − | + | − |
| ED3054 | + | − | − | − | − | + | − |
| ED3073 | n/a | − | − | − | − | + | − |

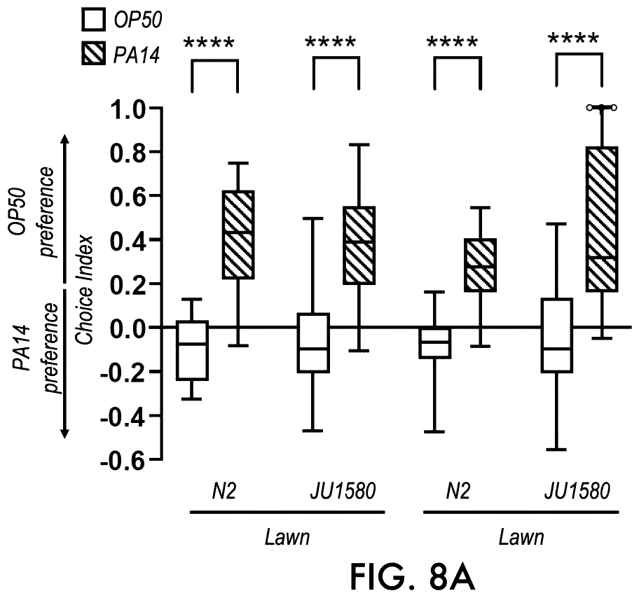

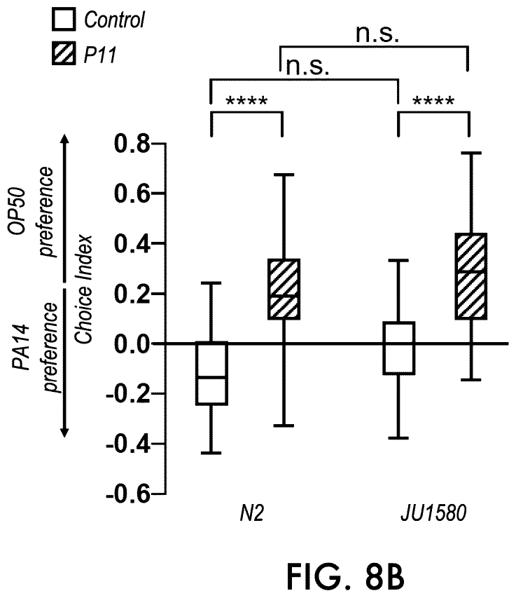

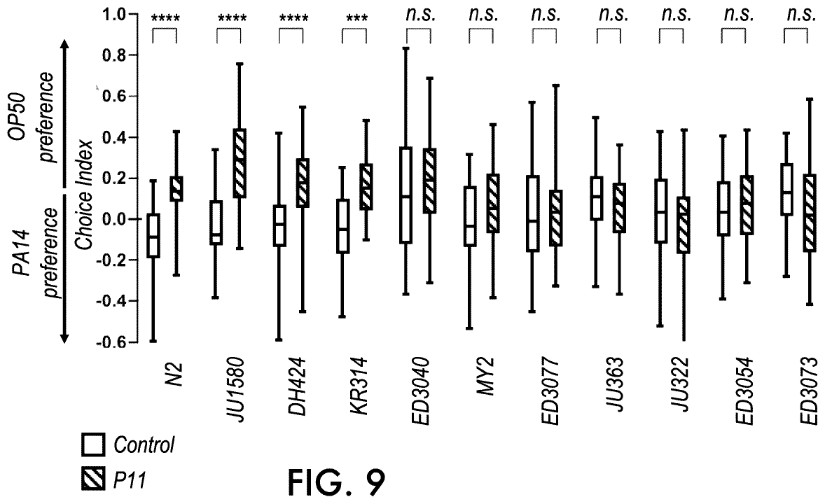

An intact copy of Cer1 is present in the wild strain JU1580, as shown by the complete coverage of the coding sequences and LTRs by de novo assembly. It was found that like N2, JU1580 animals learn to avoid P. aeruginosa both through exposure to the pathogen (FIG. 8A, left) and small RNAs (FIG. 8A, right), as well as by exposure to E. coli+P11 (see FIG. 8B).

Furthermore, trained JU1580 mothers can pass this information on to their progeny for four generations, just as N2 does. These results suggest that the mechanisms underlying transgenerational inheritance of learned pathogen avoidance via small RNAs are conserved.

In contrast to our findings with JU1580, another C. elegans strain, CB4856 (“Hawaiian”), is unable to learn to avoid P. aeruginosa after lawn or E. coli+P11 training, or to pass this information on to its progeny (F1). It was previously shown that Hawaiian does not have Cer1 inserted into its genome, but this is not the only difference between N2 and Hawaiian. CB4856 and N2 differentially survive on P. aeruginosa, and this difference is mediated by the npr-1 gene, which regulates leaving behavior in response to oxygen levels.

However, the genomic region of npr-1 in JU1580 has the “wild” SNP of npr-1, as Hawaiian does, ruling out npr-1 as the source of the difference in pathogenic learning ability. Similarly, the maco-1 gene, which is downregulated upon exposure to P. aeruginosa and is required for learned P. aeruginosa avoidance behavior, is identical between N2 and Hawaiian in the 17 nucleotides of homology to P11, suggesting that Hawaiian's inability to learn and pass on learned avoidance is not due to a lack of sequence matching between P11 small RNA and its maco-1 target.

To determine whether the presence of Cer1 correlates with the ability to learn pathogen avoidance more widely in nature, the expression of Cer1 RNA was examined via RT-PCR (see Table 1) and the presence of Cer1 GAG protein via immunofluorescence in N2, JU1580, Hawaiian, and an additional nine wild strains of C. elegans, and the ability of these wild strains to carry out P11-mediated learned avoidance of P. aeruginosa was tested.

Like N2 and JU1580, the wild strains DH424 and K4314 expressed Cer1 RNA and Cer1 GAG protein, and were able to learn P. aeruginosa avoidance after P11 training. Other strains behaved like Hawaiian, as they were unable to learn P11-induced avoidance and were defective for attraction to P. aeruginosa, none of these strains had Cer1 inserted into the genome or expressed Cer1 at appreciable levels (see, e.g., Table 1). (Although the twelfth strain, ED3040, has Cer1 inserted into its genome and expresses Cer1, it is defective for normal attraction to P. aeruginosa and does not exhibit increased avoidance upon training.)

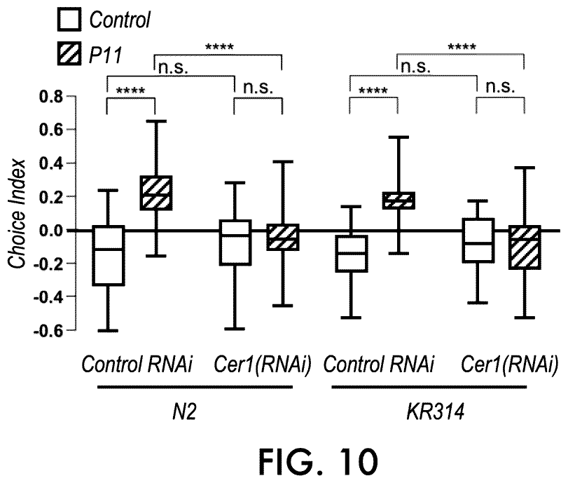

Finally, treatment of the Cer1-expressing wild strain KR314 with Cer1 RNAi abolished its P11-mediated learning (see FIG. 10). Thus, the presence and expression of Cer1 in wild strains of C. elegans largely correlates with ability to learn to avoid P. aeruginosa after small RNA-mediated training.

Thus, it is shown that information conveying pathogenic exposure status can be transferred from trained to naïve C. elegans, via capsids of Ty3/Gypsy Cer1 retrotransposon. Additionally, the transfer of this information induces memory that lasts for four additional generations, similar to training on Pseudomonas aeruginosa or its small RNA, P11. These results provide a molecular mechanism by which memory transmission might occur: the Cer1 retrotransposon expresses virus-like particles that can confer memory of learned pathogen avoidance to other individuals, and within an individual, from germline to neurons. Thus, memories of learned avoidance of pathogens can be transferred between individuals, and can induce transgenerational inheritance of the learned information.

The idea that memory can be transferred between individuals is old but controversial. Reports of horizontally-transferred memory in planarians seemed to contradict both the concept of memory storage occurring only at synapses and the strict protection of the germline from somatic changes proposed by Weismann in the late 1800s. These findings were more recently supported by an independent study in Planaria that used an automated system to reduce bias. However, planaria divide asexually, and thus the concept of a Weismann barrier might be less strict. Furthermore, no molecular mechanism for this type of memory transfer has been determined. Another example of memory transfer between individuals is from recent work in Aplysia, in which the RNA extracted from the CNS of trained animals injected into naïve animals was able to increase sensitization in a DNA methylation-dependent manner, an example of an epigenetic mechanism of memory storage, but whether this could happen in the wild or influence the behavior of progeny is unknown. The disclosed results in C. elegans suggest that the Cer1 retrotransposon enables the transfer of a memory of a pathogen from germline to nervous system, between generations, and from animal to animal.

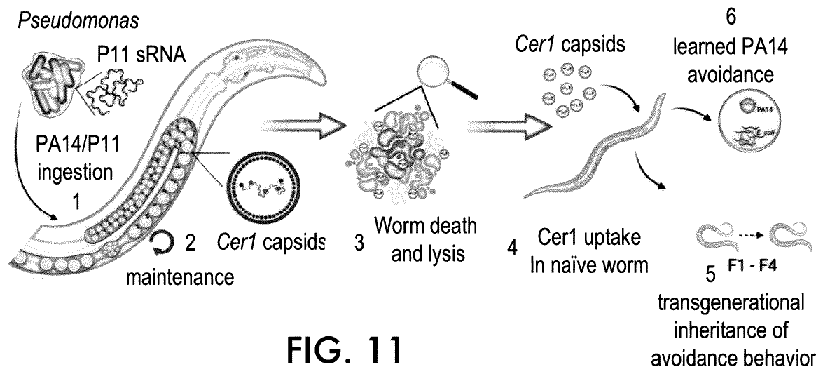

The fact that Cer1's presence in wild strains of C. elegans correlates with the ability to learn and transgenerationally inherit pathogen avoidance suggests that Cer1 itself may have enabled the acquisition of this behavior. C. elegans dies within 23 days in the presence of Pseudomonas aeruginosa, killing mothers before they have finished reproducing, which would deleteriously affect their fitness. Cer1 was previously noted to reduce fecundity in non-pathogenic conditions, but here it was found that the presence of Cer1 enables the worms to learn to avoid Pseudomonas. If naïve animals are able to take up Cer1 capsids from animals who have died and lysed, it would allow them to acquire learned avoidance without experiencing illness themselves (see FIG. 11), effectively vaccinating them against future P. aeruginosa exposure by inducing avoidance behavior.

Furthermore, as infected mothers often “bag” (die of matricide), the ability of other worms to take up Cer1 VLPs might provide them with the ability to avoid the pathogen—perhaps the first utilization of memory transfer. The ability to avoid pathogens for multiple generations could provide C. elegans that have acquired Cer1 an advantage in environments rife with pathogens.

Here it has been shown that rather than being solely deleterious, the presence of the Cer1 retrotransposon in fact may have been co-opted by C. elegans to help it survive in an environment that requires frequent encounters with pathogens. The ability of the Cer1 retrotransposon to confer a benefit to the host is surprising considering the classical nature of transposons in genomes. Transposons are highly abundant in animal genomes, and generally regarded as pernicious, mutagenic genetic elements whose mobility can lead to disease and the erosion of host fitness. Transposons incur damage to hosts on several fronts: through misregulation of host processes, such as interfering with host transcription, processing of mRNAs, and chromatin structure, or through disruption of the host genome through transposition. Consistent with other transposons, the presence of Cer1 was previously only noted to be deleterious, as its expression decreases fecundity and lifespan in non-pathogenic conditions. The finding that Cer1 is required for learned and transgenerationally-inherited PA14 avoidance behavior shows that ancient retrotransposons can be co-opted and repurposed to benefit the worm, an example of transposon-host mutualism. Since retrotransposition in C. elegans has never observed under laboratory conditions, it is likely that Cer1 mediates this acquired worm behavior independent of its potential for novel genome insertion as a retrotransposon.

While the domestication of transposons underlies some of the most critical transitions in animal evolution, the requirement for Cer1 in transgenerational learned behavior is unique in that Cer1 is an active transposon, and that Cer1 confers a behavioral ability, avoidance, on the animals. An interesting parallel arises with comparison to recent studies of Arc (of Ty3/Gypsy family origin), which showed that Arc VLPs can transmit cellular genetic material across neurons in a process that underlies synaptic plasticity in fly and mammalian brains. While C. elegans lacks a direct Arc ortholog, Cer1 is also a member of the Ty3/Gypsy family and similarly forms capsids. Cer1's role in pathogen avoidance, and specifically in the avoidance behavior step—rather than in generation or maintenance of the transgenerational memory—was surprising, given the fact that Cer1 produces VLPs in the germline; however, VLPs are also present in non-germline cells at lower abundance, perhaps suggesting at least a transient presence outside of the germline. Although it is possible that Cer1 acts like Arc, transmitting information between neurons, a more parsimonious explanation, given the abundance of Cer1 VLPs in the germline and our genetic evidence placing it upstream of daf-7 regulation in the ASI neuron, is that germline Cer1 VLPs carry host cargo to neurons, where subsequent changes in expression and activity modulate behavior.

This data suggests that Cer1 functions in a novel, dynamic germline-to-neuron signaling mechanism that may represent the co-option of retrotransposon function to improve C. elegans' survival, and its progeny's survival, in pathogenic environments. Cer1 appears to provide C. elegans immediate protection from abundant pathogenic Pseudomonas species in its environment, but also confers lasting generational benefits by communicating an adaptive immune signal of learned avoidance to its descendants. Moreover, the ability to provide memories of pathogen avoidance to neighboring worms might allow greater survival of its kin.

In some embodiments, Cer1-containing VLPs can be obtained in bulk from C. elegans using large-scale worm culture in liquid and purification of Cer1 VLPs from conditioned media using standard PEG precipitation and subsequent centrifugation.

In some embodiments, Cer1-containing VLPs from conditioned media can be fluorescently tagged using a GFP-Cer1 translational fusion protein expressed in C. elegans. Labelled Cer1-containing VLPs can be sorted from bulk secreted vesicles using flow virometry using known techniques (see, e.g., Gaudin & Barteneva, 2015, incorporated by reference herein in its entirety).

In some embodiments, an exogenous RNA-based agent can be incorporated into Cer1 VLPs by germline expression of small RNAs (miRNAs or piRNAs), or expression of foreign mRNAs to which siRNAs will be targeted using the endogenous C. elegans small RNA pathways, which are known in the art. In some embodiments, the incorporation of various small RNAs into Cer1 VLPs can then be assessed.

### Additional Examples

The human pathogen, Strongyloides stercoralis, is a soil-transmitted nematode that requires the neuronal tax-4 gene for chemosensation during the infective iL3 stage to identify host-emitted odorants (see Gang et al., 2020 PNAS). Cer1-containing VLPs that also contain asRNA directed against the tax-4 gene may be used to target the tax-4 mRNA in S. stercoralis neurons and interfere with host targeting and establishment of infection.

Meloidogyne incognita, also known as knot-root nematode, is a plant-parasitic nematode with a large host range (˜5,000 plant species infected, see Blok et al., 2008) that causes substantial crop damage (see Elling, 2013). Chemosensation is required to sense plant odorants derived from the rhizosphere to establish infection in the plant host. It is known that the neuron-expressed chemosensory genes odr-1, odr-3, tax-2, and tax-4 are required for normal M. incognita attraction to plant roots (see Shivakumara et al., 2019). Targeting these neuronal genes using Cer1-containing VLPs may limit the extent of M. incognita infection in various commercial crops.

In C. elegans, very low levels (sparse) of Cer1 are required to induce effects; it is expected that other nematodes that also use RNA interference will similarly take up and amplify the Cer1-contained message.

While the invention is described through the above-described exemplary embodiments, modifications to, and variations of, the illustrated embodiments may be made without departing from the inventive concepts disclosed herein. For example, although specific parameters, such as a specific species, etc., may be recited in relation to disclosed embodiments, within the scope of the invention, the values of all parameters may vary over wide ranges to suit different applications.

As used herein, including in the claims, the term “and/or,” used in connection with a list of items, means one or more of the items in the list, i.e., at least one of the items in the list, but not necessarily all the items in the list.

Disclosed aspects, or portions thereof, may be combined in ways not listed above and/or not explicitly claimed. In addition, embodiments disclosed herein may be suitably practiced, absent any element that is not specifically disclosed herein. Accordingly, the invention should not be viewed as being limited to the disclosed embodiments.

What is claimed is:

1. A method for delivering a RNA-based treatment, comprising:
providing a virus-like particle (VLP) containing C. elegans retrotransposon 1 (Cer1) and a heterologous RNA-based agent to an organism; and
allowing the organism to transfer the heterologous RNA-based agent to a tissue within the organism.

2. The method according to claim 1, wherein the VLP is secreted by an expressing organism.

3. The method according to claim 1, wherein the VLP is present in a lysate from a lysed organism.

4. The method according to claim 1, wherein the VLP is present in a medium.

5. The method according to claim 1, wherein the tissue comprises a neuron.

6. The method according to claim 1, wherein the tissue is free of a neuron.

7. The method according to claim 1, wherein the heterologous RNA-based agent comprises a RNA interference molecule.

8. The method according to claim 1, wherein the heterologous RNA-based agent comprises messenger RNA (mRNA).

9. The method according to claim 1, wherein the heterologous RNA-based agent comprises antisense RNA (asRNA).

10. The method according to claim 1, wherein the heterologous RNA-based agent comprises a RNA aptamer.

11. The method according to claim 1, wherein the heterologous RNA-based agent comprises a peptide.

12. The method according to claim 1, wherein the heterologous RNA-based agent comprises a molecule that is not replicated or expressed by the VLP or the organism.

13. The method according to claim 1, wherein the organism is an animal.

14. The method according to claim 1, wherein the organism is a plant.

15. The method according to claim 1, wherein the RNA-based treatment is a preventative treatment for parasitic nematodes, pathogenic nematodes, or a combination thereof.

16. The method according to claim 1, wherein the organism is allowed to transfer both the heterologous RNA-based agent and a peptide to the tissue.

17. A virus-like particle (VLP) comprising:
C. elegans retrotransposon 1 (Cer1); and
a RNA-based therapeutic agent.

18. An RNA-based therapeutic system, comprising:
a virus-like particle (VLP) according to claim 17; and
a pharmaceutically acceptable carrier.

19. A plasmid system comprising:
a first plasmid including at least one nucleotide sequence encoding Cer1 and a fluorescent protein.

20. The plasmid system according to claim 19, wherein the first plasmid also includes at least one nucleotide sequence encoding an RNA-based agent.

21. The plasmid system according to claim 19, further comprising a second plasmid that includes at least one nucleotide sequence encoding an RNA-based agent.
